SEQUENCE LISTING (2 sequences)

SEQ ID NO: 1 - DNA, 32 bp, Artificial Sequence
  primer
ctgtgaagctcgcatactctgcagataatcag

SEQ ID NO: 2 - DNA, 36 bp, Artificial Sequence
  primer
gtcagtaaagtcgaccatataaatggaacgtatcgc

(19) United States
(12) Patent Application Publication
(10) Pub. No.: US 2004/0249219 A1
(43) Pub. Date: Dec. 9, 2004

(54) Method of making teprenone

(76) Inventor: Gabriel G. Saucy, Essex Fells, NJ (US)

Correspondence Address: SHERIDAN ROSS PC, 1560 BROADWAY, SUITE 1200, DENVER, CO, 80202

(21) Appl. No.: 09/899,418

(22) Filed: Jul. 3, 2001

Related U.S. Application Data
(60) Provisional application No. 60/215,897, filed on Jul. 5, 2000.

(51) Int. Cl. C07C 45/68

(52) U.S. Cl. 568/388

(57) ABSTRACT

The invention is directed to an efficient and economical method of making teprenone. Teprenone is synthesized by converting geranylgeraniol to teprenone by a novel route. The method of synthesis can begin with geranylgeraniol obtained from a biological source such as fermentation of a microorganism capable of producing geranylgeranyl or enzymatic synthesis in a cell free system to produce predominantly the 5E isomer of teprenone. The chemical synthesis proceeds with retention of configuration such that the teprenone produced has the isomeric configuration of the geranylgeraniol starting material.

### REFERENCE TO RELATED APPLICATION

This application claims priority under 35 U.S.C. § 119(e) from U.S. Provisional Application Ser. No. 60/215,897 filed Jul. 5, 2000, entitled “Method of Making Tepranone”[sic].

### FIELD OF THE INVENTION

The present invention relates to a method of making teprenone (6,10,14,18-tetramethyl-5,9,13-nonadecatetren-2-one) starting with geranylgeraniol obtained from a biological source.

### BACKGROUND OF THE INVENTION

Inflammatory and ulcerative diseases of the gastric mucosa have increased in frequency over the past two decades affecting ten to fifteen percent of the U.S. population. The incidence of these diseases increases with age, most commonly between the ages of 25 and 75. Ulcers develop when hydrochloric acid and pepsin come in contact with the lining of the stomach and duodenum resulting in an open sore or raw area in the gastric mucosa in these areas. In order for pepsin and hydrochloric acid to cause damage to the stomach or duodenum, however, the mucous and bicarbonate layer, which coats the stomach and duodenum, must be weakened or disturbed allowing access to acid and digestive enzymes. Factors associated with breakdown of the protective mucus layer include infection with H. pylori, smoking, alcohol abuse, coffee-drinking, stress, radiation, chronic use of non-steroidal anti-inflammatory drugs (NSAIDs), and genetics.

Patients suffering from gastritis and ulcers typically experience symptoms known collectively as dyspepsia which includes abdominal pain, discomfort, bloating, fullness, nausea, heartburn, regurgitation, and belching. Dyspepsia may be persistent or recurrent, and the pain can be either localized or diffuse. For most people with severe ulcers, the most significant problem is pain and sleeplessness, which can have a dramatic and adverse impact on the quality of life. Rarely, the disease can become very serious progressing to the point of hemorrhage or perforation of the stomach or duodenum. Of the people who get ulcers, up to 15% will experience some degree of bleeding. Fortunately, the incidence of bleeding is declining with the introduction of effective treatments, but it is still one of the most common medical emergencies. Indeed, NSAID-related stomach problems are responsible for 60,000 hospital admissions and over 3,000 deaths each year in American patients.

Treatment of these diseases typically includes diet, exercise, pharmaceutical intervention, and surgery. While it is possible to alleviate some symptoms through controlled diet and exercise, the most cost effective treatment has been prevention and management with different classes of pharmaceuticals. These compounds include the antacids, antibiotics, histamine blockers, proton pump inhibitors, misoprostol, cyclooxygenase-2 inhibitors, cytoprotective agents, and most often, combinations of these drugs. These agents work by neutralizing or decreasing the amount of acid produced in the stomach, by eliminating the causative organism in the case of infections, and by directly protecting or increasing the body's natural protection of the gastric mucosa. By using these compounds together in different combinations, the destructive properties of gastric acid are decreased while the mechanisms protecting the gastric mucosa are increased, thereby providing the gastric mucosa with sufficient time to heal.

Cytoprotective agents prevent gastric mucosal injury through stimulation of gastric mucus synthesis and secretion. Cytoprotective antiulcer agents include prenyl ketones, benexate, sofalcone, cetraxate and gefarnate. Teprenone is an example of a prenyl ketone useful in the treatment and prevention of gastritis and the treatment of gastric ulcers. Teprenone increases levels of gastric mucosal hexosamine and adherent mucus thereby promoting epithelial regeneration. Studies of the efficacy of teprenone showed that teprenone significantly promoted the healing of gastric ulcers to a white scar which is indicative of a low recurrence rate of these ulcers. Additionally, teprenone used in combination with Omeprazole (a proton pump inhibitor) significantly decreased the recurrence of gastric ulcers infected with H. pylori.

Teprenone (as the brand SELBEX) is marketed by Eisai Co., Ltd. of Tokyo, Japan for the therapeutic and prophylactic treatment of gastritis, and peptic or duodenal ulcers. Increased use of teprenone, however, has suffered from the lack of a readily available supply at a cost competitive with other pharmaceuticals. Previous synthesis methods required anhydrous reaction conditions, disclosed in U.S. Pat. No. 4,814,353, or aqueous reaction conditions in the presence of certain amine catalysts disclosed in U.S. Pat. No. 3,983,175 and resulted in a racemic mixture of the 5E and 5Z isomers of teprenone (6,10,14,18-tetramethyl-5,9,13,17-nonadecatetren-2-one). Another known method for the production of teprenone from geranyllinalool is by the Carrol Reaction, e.g., as described in U.S. Pat. Nos. 4,814,353 and 5,663,461. This process does not retain the isomeric configuration of the starting materials. It is preferable to make predominately the 5E isomer as this isomer is responsible for the majority of the therapeutic activity of teprenone. Therefore, less teprenone need be administered, and fewer adverse effects will be experienced, if the teprenone is predominately composed of the 5E isomer. Additionally, both methods resulted in a costly product partly due to the expense of the starting materials. Thus, there is a need for an efficient method of making 6,10,14,18-tetramethyl-5E,9E,13E-nonadecatetren-2-one from readily available starting materials.

### SUMMARY OF THE INVENTION

The invention is directed to an efficient and economical method of making teprenone. Teprenone is synthesized by converting geranylgeraniol to produce teprenone by a novel method. The method of synthesis can begin with geranylgeraniol obtained from a biological source such as fermentation of a microorganism capable of producing geranylgeranyl or enzymatic synthesis in a cell free system to produce predominately the 5E isomer of teprenone. The chemical synthesis proceeds with the retention of configuration such that the teprenone product will have the isomeric configuration of the geranylgeraniol starting material.

In one embodiment of the invention, geranylgeraniol is reacted with an alkyl acetoacetate to form a keto ester intermediate which is then decarboxylated to form teprenone. The geranylgeraniol starting material can be converted to an alkyl halide by reaction with a halogenating agent. The alkyl halide is then contacted with the alkyl acetoacetate in the presence of a base to form the keto ester intermediate. This intermediate is then decarboxylated in the presence of an alkaline reagent. The teprenone product formed by this methodology will have the same isomeric configuration as the geranylgeraniol starting material and it is therefore possible to produce teprenone comprising close to 100% of the 5E-isomer.

In another embodiment of the invention, the geranylgeraniol starting material is produced biologically prior to conversion to teprenone. This can be done in a cell free system by reacting isopentyl diphosphate with isopentenyl diphosphate:dimethylallyl diphosphate isomerase in the presence of geranylgeranyl diphosphate synthase to form geranylgeranyl diphosphate. Alternatively, the isopentyl diphosphate can be reacted with a compound selected from dimethylallyl diphospate, geranyl diphosphate or farnesyl diphosphate to form geranylgeranyl diphosphate. The geranylgeranyl diphosphate is then dephosphorylated to obtain geranylgeraniol. The geranylgeraniol obtained from this process is predominately the 2E isomer which can be used as a starting material in the chemical synthesis of teprenone described above to produce predominately the 5E isomer of teprenone.

In another embodiment of the invention, the geranylgeraniol starting material is produced biologically by fermentation of a microorganism capable of producing geranylgeraniol. In this embodiment, the microorganism can be genetically modified to increase the geranylgeraniol production through increased enzymatic activity of enzymes involved in the production of geranylgeraniol. Additionally, the microorganism can be genetically modified to decrease the enzymatic activity of enzymes involved in the depletion of cellular resources used to produce geranylgeraniol. The geranylgeraniol obtained from this process is predominately the 2E isomer which can be used as a starting material in the chemical synthesis of teprenone described above to produce predominately the 5E isomer of teprenone.

### BRIEF DESCRIPTION OF THE DRAWINGS

FIG. 1A i-iv illustrates a diagramatic representation of the mevalonate-dependent isoprenoid biosynthetic pathway.

FIG. 1B i-iv illustrates a diagramatic representation of the mevalonate-independent isoprenoid biosynthetic pathway.

FIG. 2 shows a chemical synthesis route for the conversion of geranylgeraniol to predominately 5E,9E,13E-geranylgeranylacetone used in this method.

### DETAILED DESCRIPTION OF THE INVENTION

1. Introduction

The present invention generally relates to a method of producing teprenone from geranylgeraniol. The present invention also relates to teprenone and formulations or products containing teprenone which are produced according to the present method. The term “geranylgeranylacetone” is also referred to generically herein as “teprenone,” and as such, the two terms can be used interchangeably. The complete chemical name for geranylgeranylacetone (teprenone) is 6,10,14,18-tetramethyl-5,9,13,17-nonadecatetraen-2-one. The method of the present invention provides a novel method for the chemical synthesis of teprenone from geranylgeraniol.

Preferred sources of GGOH for the present invention are from biological production. Biological production methods include fermentation of geranylgeraniol such as is described below in section 3. Another biological production method is enzymatic production of geranylgeraniol such as is described below in section 4. As used herein, “biological production” and similar terms mean production of a chemical in man-made systems using biological organisms or molecules, such as by fermentation or by an in vitro enzymatic reaction process. “Biological production” does not include processes such as recovery and purification of a chemical from naturally occurring sources, such as plants.

The present invention utilizing geranylgeraniol from a biological source produces predominately the 5E isomer of teprenone by converting the biologically produced geranylgeraniol to teprenone via a chemical synthesis pathway which retains the isomeric configuration of the starting material. The geranylgeraniol starting material is produced biologically resulting in the 2E isomer of geranylgeraniol. This biological production is accomplished by fermentation of a microorganism capable of producing geranylgeraniol or via a cell-free enzymatic methodology to produce isomerically pure 2E geranylgeraniol. These two methods of production result in a geranylgeraniol starting material comprising a high concentration of 3,7,11,15-tetramethyl-2E,6,10,14-hexadecatetraene, the 2E-isomer of geranylgeraniol. Such biological methods of production can produce geranylgeraniol having at least about 75% of the 2E-isomer, more preferably, at least about 90% of this 2E-isomer, and even more preferably, at least about 95% of the 2E-isomer.

2. Production of Geranylgeraniol by Chemical Synthesis

Geranylgeraniol for use in the present invention can be obtained from any known source. One method of chemical synthesis of all-trans GGOH is described in Mu, Y. and Gibbs, R., Terahedron Letters, 36 (32), 5669-72 (1995), which is incorporated herein by reference. This synthesis route begins by coupling the farnesyl bromide starting material with the dianion derived from ethyl acetoacetate to produce a β-ketoester. The carboxyl of the ketone group in the beta position is then converted to a vinyl triflate which is then coupled with methaneboronic acid in an ether dioxane solvent employing both Ag₂) and K₃PO₄ bases in the palladium-catalyzed methylation to give all trans-ethyl geranylgeranoate. The geranoate is then reduced with DIBAL to form all-trans-geranylgeraniol.

Another synthetic method is described in U.S. Pat. No. 4,169,157 to Kijima et al., incorporated herein by reference, wherein the twenty carbon aliphatic halide is coupled to ethyl acetoacetate in the presence of a condensation agent. The condensation product is treated with an alkali reagent to allow ester cleavage and decarboxylation to form geranylgeraniol.

Another method of obtaining the all-trans geranylgeraniol is the crystallization procedure described in U.S. Pat. No. 5,663,461, incorporated herein by reference. In this procedure, the ester precursor of geranylgeraniol is produced by any non-stereoselective means and the resulting mixture of geometric isomers is subjected to crystallization in a suitable solvent. The mixture is cooled slowly and a seed crystal of the desired isomer is added. The resulting crystal is filtered, resuspended and converted to geranylgeraniol by hydrolysis.

3. Production of Geranylgeraniol by Fermentation

One method of biological production of geranylgeraniol is by culturing a microorganism in a fermentation medium to produce geranylgeraniol. Various microorganisms and methods for the production of geranylgeraniol by fermentation are described in the Examples section of WO 00/01650, published on Jan. 13, 2000, which Examples are incorporated herein by reference in their entirety.

3.1 Production Microorganism

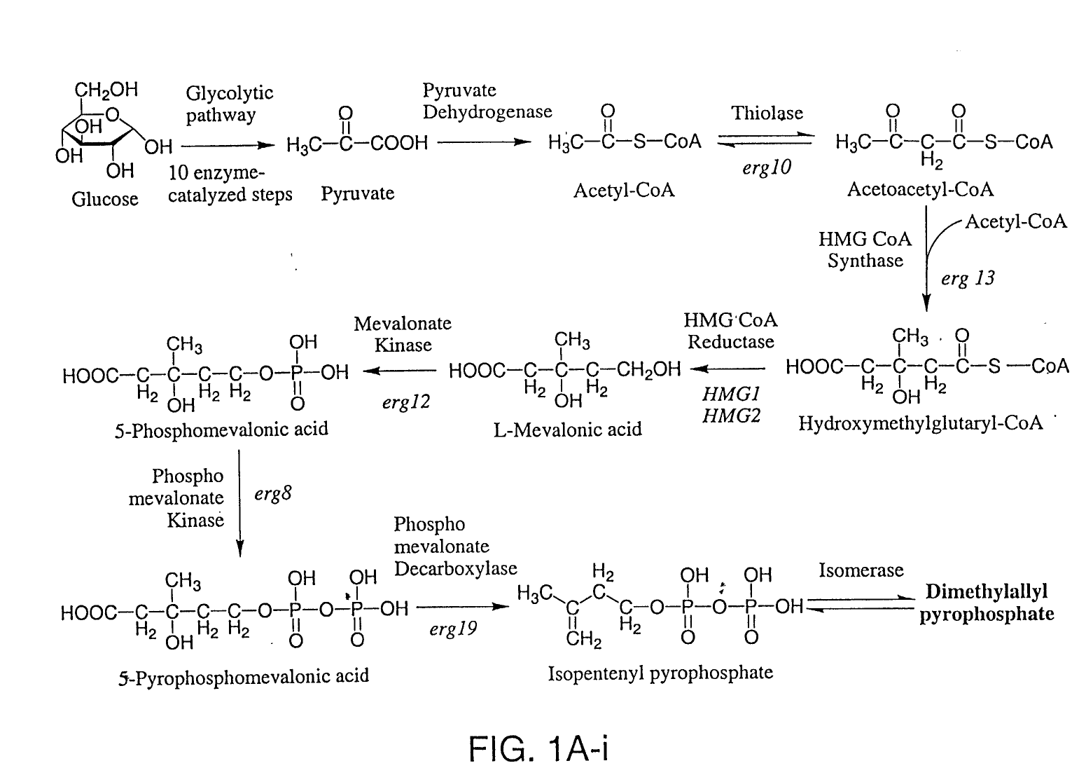

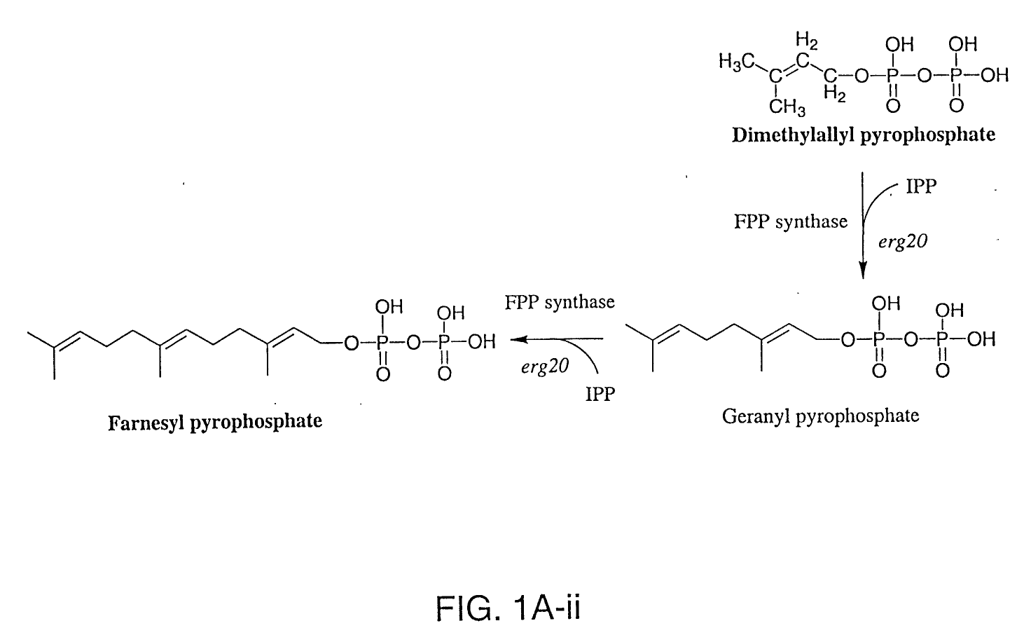

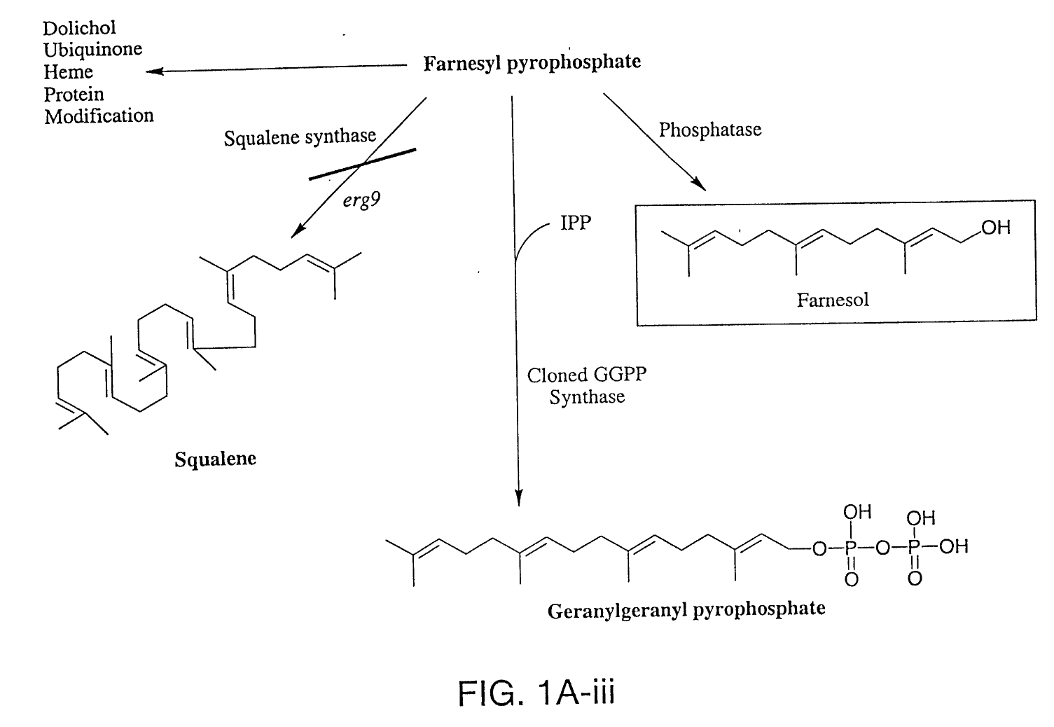

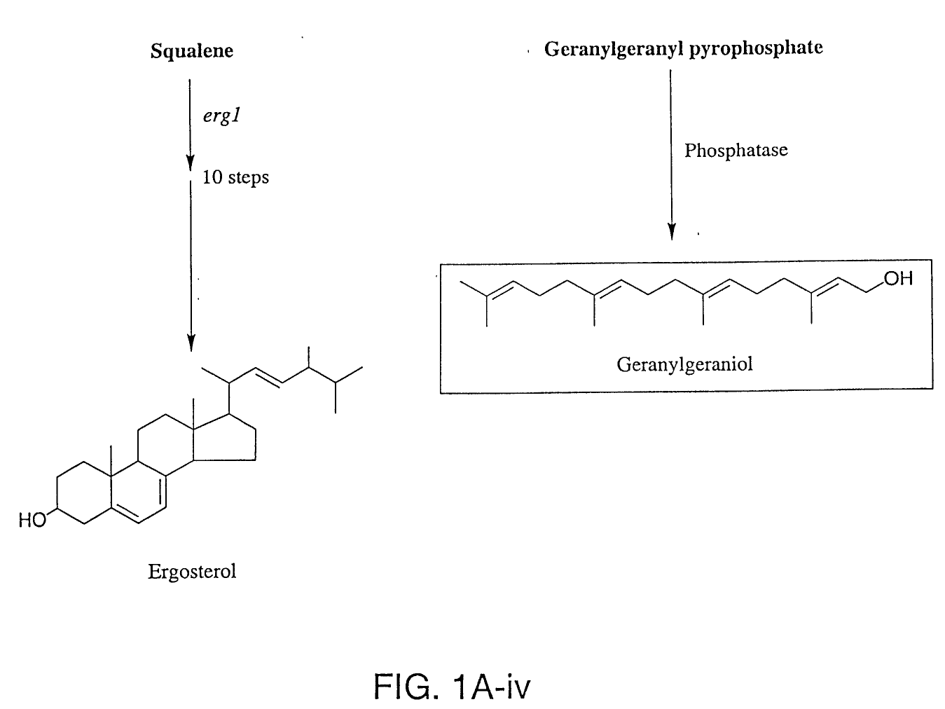

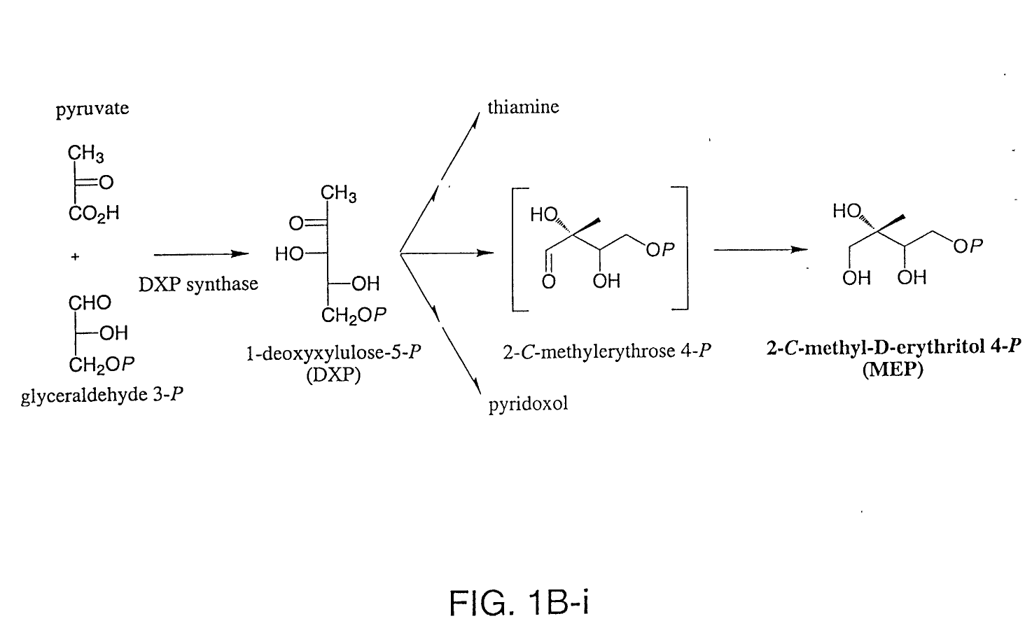

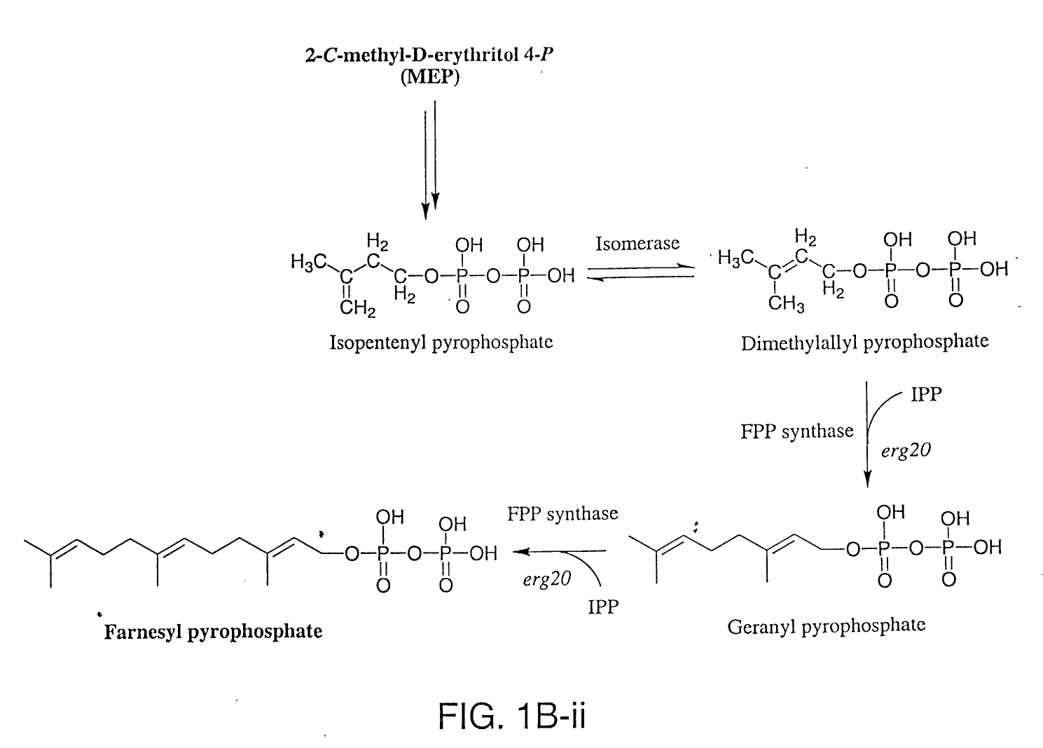

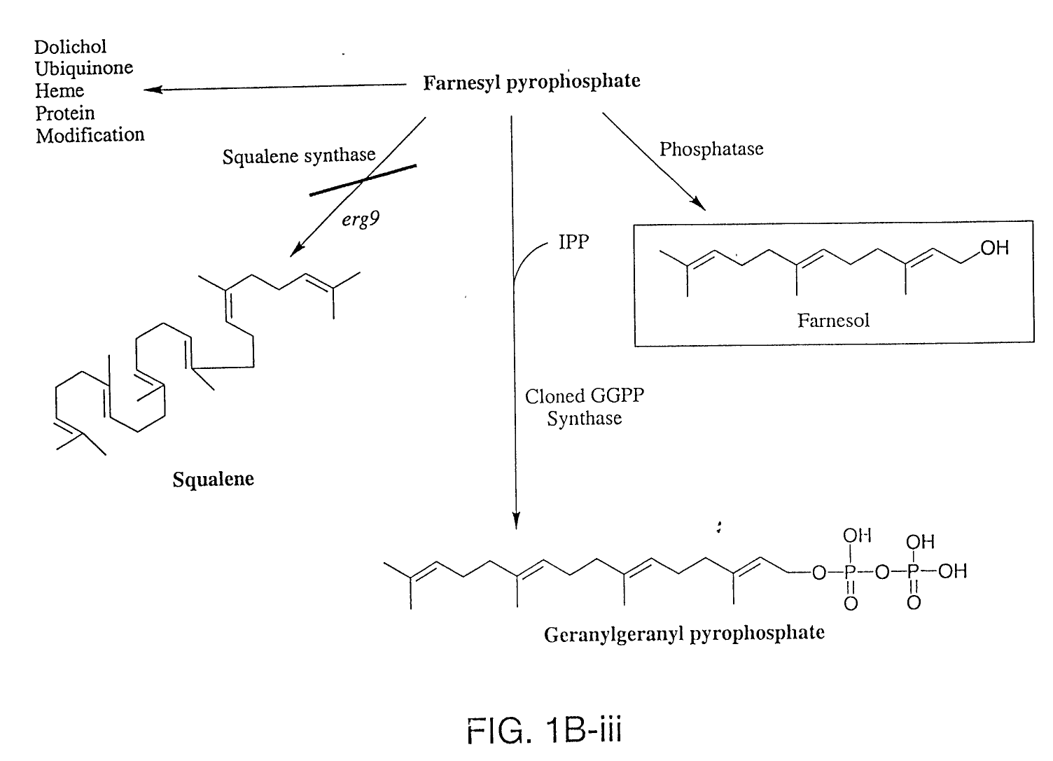

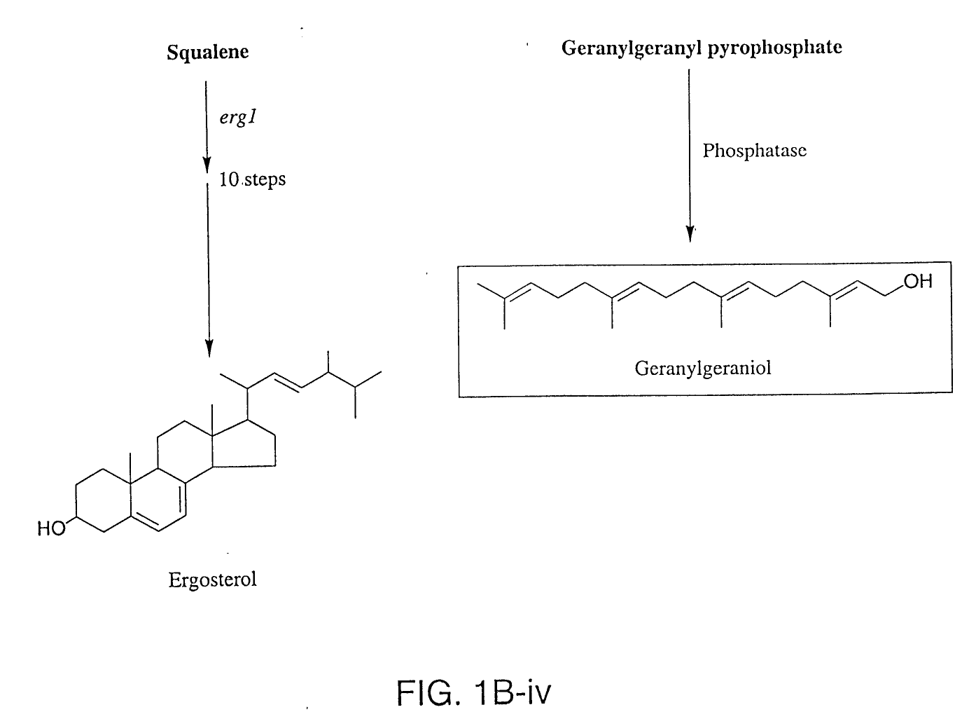

Suitable biological systems for producing GG include prokaryotic and eukaryotic cell cultures and cell-free enzymatic systems. Preferred biological systems include fungal, bacterial and microalgal systems. More preferred biological systems are fungal cell cultures, more preferably a yeast cell culture, and most preferably a Saccharomyces cerevisiae cell culture. Fungi are preferred since they have a long history of use in industrial processes and can be manipulated by both classical microbiological and genetic engineering techniques. Yeast, in particular, are well-characterized genetically. Indeed, the entire genome of S. cerevisiae has been sequenced, and the genes coding for enzymes in the isoprenoid pathway have already been cloned. Also, S. cerevisiae grows to high cell densities, and amounts of squalene and ergosterol (see FIG. 1) up to 16% of cell dry weight have been reported in genetically-engineered strains. For a recent review of the isoprenoid pathway in yeast, see Parks and Casey, Annu. Rev. Microbiol. 49:95-116 (1995).

The preferred prokaryote is E. coli. E. coli is well established as an industrial microorganism used in the production of metabolites (amino acids, vitamins) and several recombinant proteins. The entire E. coli genome has also been sequenced, and the genetic systems are highly developed. As mentioned above, E. coli uses the mevalonate-independent pathway for synthesis of IPP. The E. coli dxs, dxr, idi, and ispA genes, encoding D-1-deoxyxylulose 5-phosphate synthase, D-1-deoxyxylulose 5-phosphate reductoisomerase, IPP isomerase (IDI), and farnesyl diphosphate (FPP) synthase, respectively, have been cloned and sequenced (Fujisaki, et. al, J. Biochem. 108, 995-1000 (1990); Lois et al., Proc. Natl. Acad. Sci. USA, 95, 2105-2110 (1998); Hemmi et al., J. Biochem., 123, 1088-1096 (1998)).

Preferred microalga for use in the present invention include Chlorella and Prototheca.

Suitable organisms useful in producing farnesol and GG are available from numerous sources, such as the American Type Culture Collection (ATCC), Rockville, Md., Culture Collection of Algae (UTEX), Austin, Tx., the Northern Regional Research Laboratory (NRRL), Peoria, Ill. and the E. coli Genetic Stock Center (CGSC), New Haven, Conn. In particular, there are culture collections of S. cerevisiae that have been used to study the isoprenoid pathway which are available from, e.g., Jasper Rine at the University of California, Berkeley, Calif. and from Leo Parks at North Carolina State University, Raleigh, N.C.

Preferably the cells used in the cell culture are genetically modified to increase the yield of farnesol or GG. Cells may be genetically modified by genetic engineering techniques (i.e., recombinant technology), classical microbiological techniques, or a combination of such techniques and can also include naturally occurring genetic variants. Some of such techniques are generally disclosed, for example, in Sambrook et al., 1989, Molecular Cloning: A Laboratory Manual, Cold Spring Harbor Labs Press. The reference Sambrook et al., ibid., is incorporated by reference herein in its entirety. A genetically modified microorganism can include a microorganism in which nucleic acid molecules have been inserted, deleted or modified (i.e., mutated; e.g., by insertion, deletion, substitution, and/or inversion of nucleotides), in such a manner that such modifications provide the desired effect of increased yields of farnesol or GG within the microorganism or in the culture supernatant. As used herein, genetic modifications which result in a decrease in gene expression, in the function of the gene, or in the function of the gene product (i.e., the protein encoded by the gene) can be referred to as inactivation (complete or partial), deletion, interruption, blockage or down-regulation of a gene. For example, a genetic modification in a gene which results in a decrease in the function of the protein encoded by such gene, can be the result of a complete deletion of the gene (i.e., the gene does not exist, and therefore the protein does not exist), a mutation in the gene which results in incomplete or no translation of the protein (e.g., the protein is not expressed), or a mutation in the gene which decreases or abolishes the natural function of the protein (e.g., a protein is expressed which has decreased or no enzymatic activity). Genetic modifications which result in an increase in gene expression or function can be referred to as amplification, overproduction, overexpression, activation, enhancement, addition, or up-regulation of a gene. Addition of cloned genes to increase gene expression can include maintaining the cloned gene(s) on replicating plasmids or integrating the cloned gene(s) into the genome of the production organism. Furthermore, increasing the expression of desired cloned genes can include operatively linking the cloned gene(s) to native or heterologous transcriptional control elements.

3.1.1 Squalene Synthase Modifications

Embodiments of the present invention include biological production of farnesol or GG by culturing a microorganism, preferably yeast, which has been genetically modified to modulate the activity of one or more of the enzymes in its isoprenoid biosynthetic pathway. In one embodiment, a microorganism has been genetically modified by decreasing (including eliminating) the action of squalene synthase activity (see FIG. 1). For instance, yeast erg9 mutants that are unable to convert mevalonate into squalene, and which accumulate farnesol, have been produced. Karst and Lacroute, Molec. Gen. Genet., 154 269-277 (1977); U.S. Pat. No. 5,589,372. As used herein, reference to erg9 mutant or mutation generally refers to a genetic modification that decreases the action of squalene synthase, such as by blocking or reducing the production of squalene synthase, reducing squalene synthase activity, or inhibiting the activity of squalene synthase, which results in the accumulation of farnesyl diphosphate (FPP) unless the FPP is otherwise converted to another compound, such as farnesol by phosphatase activity. Blocking or reducing the production of squalene synthase can include placing the ERG9 gene under the control of a promoter that requires the presence of an inducing compound in the growth medium. By establishing conditions such that the inducer becomes depleted from the medium, the expression of ERG9 (and therefore, squalene synthase synthesis) could be turned off. Also, some promoters are turned off by the presence of a repressing compound. For example, the promoters from the yeast CTR3 or CTR1 genes can be repressed by addition of copper. Blocking or reducing the activity of squalene synthase could also include using an excision technology approach similar to that described in U.S. Pat. No. 4,743,546, incorporated herein by reference. In this approach, the ERG9 gene is cloned between specific genetic sequences that allow specific, controlled excision of the ERG9 gene from the genome. Excision could be prompted by, for example, a shift in the cultivation temperature of the culture, as in U.S. Pat. No. 4,743,546, or by some other physical or nutritional signal. Such a genetic modification includes any type of modification and specifically includes modifications made by recombinant technology and by classical mutagenesis. Inhibitors of squalene synthase are known (see U.S. Pat. No. 4,871,721 and the references cited in U.S. Pat. No. 5,475,029) and can be added to cell cultures. In another embodiment, an organism having the mevalonate-independent pathway of isoprenoid biosynthesis (such as E. coli) is genetically modified so that it accumulates FPP and/or farnesol. For example, decreasing the activity of octaprenyl pyrophosphate synthase (the product of the ispB gene) would be expected to result in FPP accumulation in E. coli. (Asai, et al., Biochem. Biophys. Res. Comm. 202, 340-345 (1994)). The action of a phosphatase could further result in farnesol accumulation in E. coli.

Yeast strains need ergosterol for cell membrane fluidity, so mutants blocked in the ergosterol pathway, such as erg9 mutants, need extraneous ergosterol or other sterols added to the medium for the cells to remain viable. The cells normally cannot utilize this additional sterol unless grown under anaerobic conditions. Therefore, a further embodiment of the present invention is the use of a yeast in which the action of squalene synthase is reduced, such as an erg9 mutant, and which takes up exogenously supplied sterols under aerobic conditions. Genetic modifications which allow yeast to utilize sterols under aerobic conditions are demonstrated below in the Examples section of WO 00/01650 and are also known in the art. For example, such genetic modifications include upc (uptake control mutation which allows cells to take up sterols under aerobic conditions) and hem1 (the HEM1 gene encodes aminolevulinic acid synthase which is the first committed step to the heme biosynthetic pathway, and hem1 mutants are capable of taking up ergosterol under aerobic conditions following a disruption in the ergosterol biosynthetic pathway, provided the cultures are supplemented with unsaturated fatty acids). Yeast strains having these mutations can be produced using known techniques and also are available from, e.g., Dr. Leo Parks, North Carolina State University, Raleigh, N.C. Haploid cells containing these mutations can be used to generate other mutants by genetic crosses with other haploid cells. Also, overexpression of the SUT1 (sterol uptake) gene can be used to allow for uptake of sterols under aerobic conditions. The SUT1 gene has been cloned and sequenced. Bourot and Karst, Gene, 165: 97-102 (1995).

In a further embodiment, microorganisms of the present invention can be used to produce farnesol and/or GG by culturing microorganisms in the presence of a squalene synthase inhibitor. In this manner, the action of squalene synthase is reduced. Squalene synthase inhibitors are known to those skilled in the art. (See, for example, U.S. Pat. No. 5,556,990.)

3.1.2 HMG-CoA Reductase Modifications

A further embodiment of the present invention is the use of a microorganism which has been genetically modified to increase the action of HMG-CoA reductase. It should be noted that reference to increasing the action of HMG-CoA reductase and other enzymes discussed herein refers to any genetic modification in the microorganism in question which results in increased functionality of the enzymes and includes higher activity of the enzymes, reduced inhibition or degradation of the enzymes and overexpression of the enzymes. For example, gene copy number can be increased, expression levels can be increased by use of a promoter that gives higher levels of expression than that of the native promoter, or a gene can be altered by genetic engineering or classical mutagenesis to increase the activity of an enzyme. One of the key enzymes in the mevalonate-dependent isoprenoid biosynthetic pathway is HMG-CoA reductase which catalyzes the reduction of 3-hydroxy-3-methylglutaryl-coenzyme A (HMG-CoA). This is the primary rate-limiting and first irreversible step in the pathway, and increasing HMG-CoA reductase activity leads to higher yields of squalene and ergosterol in a wild-type strain of S. cerevisiae, and farnesol in an erg9 strain. One mechanism by which the action of HMG-CoA reductase can be increased is by reducing inhibition of the enzyme, by either genetically modifying the enzyme or by modifying the system to remove the inhibitor. For instance, both sterol and non-sterol products of the isoprenoid pathway feedback to inhibit this enzyme (see, e.g., Parks and Casey, Annu. Rev. Microbiol. 49:95-116 (1995). Alternatively or in addition, the gene(s) coding for HMG-CoA reductase can be altered by genetic engineering or classical mutagenesis techniques to decrease or prevent inhibition. Also, the action of HMG-CoA reductase can be increased by increasing the gene copy number, by increasing the level of expression of the HMG-CoA reductase gene(s), or by altering the HMG-CoA reductase gene(s) by genetic engineering or classical mutagenesis to increase the activity of the enzyme. See U.S. Pat. No. 5,460,949, the entire contents of which are incorporated herein by reference. For example, truncated HMG-CoA reductases have been produced in which the regulatory domain has been removed and the use of gene copy numbers up to about six also gives increased activity. Id. See also, Downing et al., Biochem. Biophys. Res. Commun., 94,974-79 (1980) describing two yeast mutants having increased levels of HMG-CoA reductase. Two isozymes of HMGCoA reductase, encoded by the HMG1 and HMG2 genes, exist in S. cerevisiae. The activity of these two isozymes is regulated by several mechanisms including regulation of transcription, regulation of translation, and for Hmg2p, degradation of the enzyme in the endoplasmic reticulum (Hampton and Rine, 1994; Donald, et. al. 1997). In both Hmg1p and Hmg2p, the catalytic domain resides in the carboxy terminal portion of the enzyme, while the regulatory domain resides in the membrane spanning NH₂-terminal region. It has been shown that overexpression of just the catalytic domain of Hmg1p in S. cerevisiae increases carbon flow through the isoprenoid pathway, resulting in overproduction of squalene (Saunders, et. al. 1995; Donald, et. al., 1997). The present inventors have expressed the catalytic domain of the S. cerevisiae Hmg2p in strains having a normal (i.e., unblocked) isoprenoid pathway and observed a significant increase in the production of squalene. Furthermore, overexpression of the catalytic domain of Hmg2p resulted in increased farnesol production in an erg9 mutant, and increased farnesol and GG production in an erg9 mutant overexpressing GGPP synthase, grown in fermentors.

3.1.3 GGPP Synthase Modifications

A further embodiment of the present invention is the use of a microorganism which has been genetically modified to increase the action of GGPP synthase. Genes coding for this enzyme from a variety of sources, including bacteria, fungi, plants, mammals, and archaebacteria, have been identified. See, Brinkhaus et al., Arch. Biochem. Biophys., 266, 607-612 (1988); Carattoli et al., J. Biol. Chem., 266, 5854-59 (1991); Chen et al., J. Biol. Chem., 268, 11002-11007 (1993); Dogbo et al., Biochim. Biophys. Acta, 920, 140-148 (1987); Jiang et al., J. Biol. Chem., 270, 21793-99 (1995); Kuntz al., Plant J., 2, 25-34 (1992); Laferriere, et al., Biochim. Biophys. Acta, 1077, 167-72 (1991); Math et al., Proc. Natl. Acad. Sci. USA, 89, 6761-64 (1992); Ohnuma et al., J. Biol. Chem., 269, 14792-97 (1994); Sagami et al., Arch. Biochem. Biophys., 297, 314-20 (1992); Sagami et al., J. Biol. Chem., 269, 20561-66 (1994); Sandmann et al., J. Photochem. Photobiol. B: Biol., 18,245-51 (1993); Scolnik et al., Plant Physiol., 104, 1469-70 (1994); Tachibana et al., Biosci. Biotech. Biochem., 7, 1129-33 (1993); Tachibana et al., J. Biochem., 114, 389-92 (1993); Wiedemann et al., Arch. Biochem. Biophys., 306, 152-57 (1993). Some organisms have a bifunctional enzyme which also serves as an FPP synthase, so it is involved in the overall conversion of IPP and DMAPP to FPP to GGPP (see FIG. 1). Some enzymes, such as those found in plants, have relaxed specificity, converting IPP and DMAPP to GGPP (see FIG. 1). Genetic modifications of GGPP synthase, as used herein, encompass engineering a monofunctional GGPP synthase or a bifunctional FPP/GGPP synthase to enhance the GGPP synthase activity component of the enzyme. A preferred GGPP synthase gene is the BTS1 gene from S. cervisiae. The BTS1 gene and its isolation are described in Jiang et al., J. Biol. Chem., 270, 21793-99 (1995) and U.S. Pat. No. 5,912,154, the complete disclosure of which incorporated herein by reference. However, GGPP synthases of other hosts can be used, and the use of the bifunctional GGPP synthases may be particularly advantageous in terms of channeling carbon flow through FPP to GGPP, thereby avoiding loss of FPP to competing reactions in the cell.

In further embodiments of the invention, in addition to the modifications of GGPP synthase described above, the wild type GGPP synthase is eliminated from the production organism. This would serve, for example, to eliminate competition between the modified GGPP synthase and the wild type enzyme for the substrates, FPP and IPP. Deletion of the wild-type gene encoding GGPP synthase could also improve the stability of the cloned GGPP synthase gene by removing regions of high genetic sequence homology, thereby avoiding potentially detrimental genetic recombination.

3.1.4 FPP Synthase Modifications

A further embodiment of the present invention is the use of a microorganism which has been genetically modified to increase the action of FPP synthase .

Genes coding for this enzyme from a variety of sources have been identified. See, Anderson et al., J. Biol. Chem., 264, 19176-19184 (1989); Attucci, et al., Arch. Biochem. Biophys., 321, 493-500 (1995); Cane et al., J. Am. Chem. Soc., 105, 122-124 (1983); Chambon et al., Current Genetics, 18, 41-46 (1990);, Chambon et al., Lipids, 26, 633-36 (1991); Chen al., Protein Science, 3, 600-607 (1994); Davisson, et al., J. Am. Chem. Soc., 115, 1235-45 (1993); Ding et al., Biochem. J., 275, 61-65 (1991); Hugueney et al., FEBS Letters, 273, 235-38 (1990); Joly et al., J. Biol. Chem., 268, 26983-89 (1993); Koyama al., J. Biochem., 113,355-63 (1993); Sheares, et al., Biochem., 28, 8129-35 (1989); Song et al., Proc. Natl. Acad. Sci. USA, 91, 3044-48 (1994); Spear et al., J. Biol. Chem., 267, 14662-69 (1992); Spear et al., J. Biol. Chem., 269, 25212-18 (1994). Anderson et al., J. Biol. Chem., 264, 19176-19184 (1989) reported a 2-3 fold overexpression of FPP synthase with the S. cerevisiae gene in a yeast shuttle vector.

It has been surprisingly found that overexpression of FPP synthase did not lead to an increase in farnesol production, but unexpectedly lead to an increase in the production of GG in the absence of any overexpression of GGPP synthase.

In further embodiments of the invention, in addition to the over-expression of FPP synthase described above, the wild type FPP synthase is eliminated from the production organism. This would serve, for example, to eliminate competition between the modified FPP synthase and the wild type enzyme for the substrates, IPP, DMAPP and GPP. Deletion of the wild-type gene encoding FPP synthase could also improve the stability of the cloned FPP synthase gene by removing regions of high genetic sequence homology, thereby avoiding potentially detrimental genetic recombination.

3.1.5. Phosphatase Modifications

A further embodiment of the present invention is the use of a microorganism which has been genetically modified to increase phosphatase action to increase conversion of FPP to farnesol or GGPP to GG. For example, both S. cerevisiae and E. coli contain numerous phosphatase activities. By testing several phosphatases for efficient dephosphorylation of FPP or GGPP, one could select an appropriate phosphatase and express the gene encoding this enzyme in a production organism to enhance farnesol or GG production. Examples of phosphatases from S. cerevisiae that could be modified include the phosphatases coded by the DPP1 and LPP1 genes. Both Dpp1 and Lpp1 phosphatases have been shown to possess isoprenoid phosphatase activity (Faulkner et al. 1999. J. Biol. Chem. 274:14831-14837). Increasing the action of these phosphatases would promote the formation of isoprenoid alcohols. In addition to (or instead of) increasing the action of a desired phosphatase to enhance farnesol or GG production, one could eliminate, through genetic means, undesirable phosphatase activities. For example, one could eliminate through mutation the activity of a phosphatase that specifically acts on FPP, so that the FPP that was spared would be available for conversion to GGPP and subsequently GG. Decreasing the action of these phosphatases may allow more FPP to be converted to GGPP.

3.1.6 Additional Genetic Modifications

Modifications of Other Isoprenoid Pathway Enzymes.

Modifications that can be made to increase the action of HMGCoA reductase, GGPP synthase and phosphatases are described above. Modification of the action of isoprenoid pathway enzymes is not limited to those specific examples, and similar strategies can be applied to modify the action of other isoprenoid pathway enzymes such as acetoacetyl Co-A thiolase, HMG-CoA synthase, mevalonate kinase, phosphomevalonate kinase, phosphomevalonate decarboxylase, IPP isomerase, farnesyl pyrophosphate synthase or D-1-deoxyxylulose 5-phosphate synthase D-1-deoxyxylulose 5-phosphate reductoisomerase.

Engineering of Central Metabolism to Increase Precursor Supply to the Isoprenoid Pathway.

In organisms having the mevalonate-dependent isoprenoid pathway, the biosynthesis of farnesol or GG begins with acetyl CoA (refer to FIG. 1). One embodiment of the present invention is genetic modification of the production organism such that the intracellular level of acetyl CoA is increased, thereby making more acetyl CoA available for direction to the isoprenoid pathway (and hence to farnesol and/or GG). For example, the supply of acetyl CoA can be increased by increasing the activity of the pyruvate dehydrogenase complex. The supply of acetyl CoA can be further increased by increasing the level of pyruvate in the cell by increasing the activity of pyruvate kinase. In organisms having the mevalonate-independent isoprenoid pathway, the biosynthesis of isoprenoids begins with pyruvate and glyceraldehyde 3-phosphate. The supply of pyruvate and glyceraldehyde 3-phosphate available for isoprenoid biosynthesis can be increased by increasing the action of pyruvate kinase and triophosphate isomerase, respectively.

The examples above are provided only to illustrate the concept of engineering central metabolism for the purpose of increasing production of isoprenoid compounds, and are not an exhaustive list of approaches that can be taken. Numerous other strategies could be successfully applied to achieve this goal.

Blocking Pathways that Compete for FPP or GGPP.

In yeast, FPP is a branch point intermediate leading to the biosynthesis of sterols, heme, dolichol, ubiquinone, GGPP and farnesylated proteins. In E. coli, FPP serves as the substrate for octaprenyl pyrophosphate synthase in the pathway leading to ubiquinone. In bacteria that synthesize carotenoids, such as Erwina uredovora, FPP is converted to GGPP by GGPP synthase in the first step leading to the carotenoids. To increase the production of farnesol or GG, it is desirable to inactivate genes encoding enzymes that use FPP or GGPP as substrate, or to reduce the activity of the enzymes themselves, either through mutation or the use of specific enzyme inhibitors (as was discussed above for squalene synthase). In S. cerevisiae, for example, it may be advantageous to inactivate the first step in the pathway from FPP to heme, in addition to inactivating ERG9. As discussed earlier, in E. coli, partial or complete inactivation of the octaprenyl pyrophosphate synthase could increase the availability of FPP for conversion of farnesol. Finally, in bacteria that produce carotenoids, such as Erwina uredovora, elimination of GGPP synthase can increase the level of FPP for conversion of farnesol, while inactivating or reducing the activity of phytoene synthase (the crtB gene product) can increase the level of GGPP available for conversion to GG.

It is possible that blocking pathways leading away from FPP or GGPP could have negative effects on the growth and physiology of the production organism. It is further contemplated that additional genetic modifications required to offset these complications can be made. The isolation of mutants of S. cerevisiae that are blocked in the isoprenoid pathway and take up sterols under aerobic conditions, as described above, illustrates that compensating mutations can be obtained that overcome the effects of the primary genetic modifications.

Isolation of Production Strains that are Resistant to Farnesol or GG

In the Examples section of WO 00/01650, production of high levels of farnesol and GG by genetically modified strains of S. cerevisiae is described. It is recognized that as further increases in farnesol or GG production are made, these compounds may reach levels that are toxic to the production organism. Indeed, product toxicity is a common problem encountered in biological production processes. However,just as common are the genetic modifications made by classical methods or recombinant technology that overcome product toxicity. The present invention anticipates encountering product toxicity. Thus a further embodiment of this invention is the isolation of mutants with increased resistance to farnesol and/or GG.

Isolation of Production Organisms with Improved Growth Properties.

One effect of blocking the isoprenoid pathway in S. cerevisiae is that the mutant organisms (in the present invention, erg9 mutants) grow more slowly than their parent (unblocked) strains, despite the addition of ergosterol to the culture medium. That the slower growth of the erg9 mutants is due to the block at erg9 is illustrated by the fact that repairing the erg9 mutation restores the growth rate of the strain to about that of the wild-type parent. The slower growth of the erg9 mutants could be due to differences related to growing on exogenously supplied ergosterol vs. ergosterol synthesized in the cell, or could be due to other physiological factors. One embodiment of the present invention is to isolate variants of the farnesol or GG producing strains with improved growth properties. This could be achieved, for example by continuous culture, selecting for faster growing variants. Such variants could occur spontaneously or could be obtained by classical mutagenesis or molecular genetic approaches.

3.2 Fermentation Media and Conditions

In the method for production of farnesol or GG, a microorganism having a genetic modification, as discussed above is cultured in a fermentation medium for production of farnesol or GG. An appropriate, or effective, fermentation medium refers to any medium in which a genetically modified microorganism of the present invention, when cultured, is capable of producing farnesol or GG. Such a medium is typically an aqueous medium comprising assimilable carbon, nitrogen and phosphate sources. Such a medium can also include appropriate salts, minerals, metals and other nutrients. In addition, when an organism which is blocked in the ergosterol pathway and requires exogenous sterols, the fermentation medium must contain such exogenous sterols. Appropriate exemplary media are shown in the discussion below and in the Examples section of WO 00/01650. It should be recognized, however, that a variety of fermentation conditions are suitable and can be selected by those skilled in the art.

Sources of assimilable carbon which can be used in a suitable fermentation medium include, but are not limited to, sugars and their polymers, including, dextrin, sucrose, maltose, lactose, glucose, fructose, mannose, sorbose, arabinose and xylose; fatty acids; organic acids such as acetate; primary alcohols such as ethanol and n-propanol; and polyalcohols such as glycerine. Preferred carbon sources in the present invention include monosaccharides, disaccharides, and trisaccharides. The most preferred carbon source is glucose.

The concentration of a carbon source, such as glucose, in the fermentation medium should promote cell growth, but not be so high as to repress growth of the microorganism used. Typically, fermentations are run with a carbon source, such as glucose, being added at levels to achieve the desired level of growth and biomass, but at undetectable levels (with detection limits being about <0.1 g/l). In other embodiments, the concentration of a carbon source, such as glucose, in the fermentation medium is greater than about 1 g/L, preferably greater than about 2 g/L, and more preferably greater than about 5 g/L. In addition, the concentration of a carbon source, such as glucose, in the fermentation medium is typically less than about 100 g/L, preferably less than about 50 g/L, and more preferably less than about 20 g/L. It should be noted that references to fermentation component concentrations can refer to both initial and/or ongoing component concentrations. In some cases, it may be desirable to allow the fermentation medium to become depleted of a carbon source during fermentation.

Sources of assimilable nitrogen which can be used in a suitable fermentation medium include, but are not limited to, simple nitrogen sources, organic nitrogen sources and complex nitrogen sources. Such nitrogen sources include anhydrous ammonia, ammonium salts and substances of animal, vegetable and/or microbial origin. Suitable nitrogen sources include, but are not limited to, protein hydrolysates, microbial biomass hydrolysates, peptone, yeast extract, ammonium sulfate, urea, and amino acids. Typically, the concentration of the nitrogen sources, in the fermentation medium is greater than about 0.1 g/L, preferably greater than about 0.25 g/L, and more preferably greater than about 1.0 g/L. Beyond certain concentrations, however, the addition of a nitrogen source to the fermentation medium is not advantageous for the growth of the microorganisms. As a result, the concentration of the nitrogen sources, in the fermentation medium is less than about 20 g/L, preferably less than about 10 g/L and more preferably less than about 5 g/L. Further, in some instances it may be desirable to allow the fermentation medium to become depleted of the nitrogen sources during fermentation.

The effective fermentation medium can contain other compounds such as inorganic salts, vitamins, trace metals or growth promoters. Such other compounds can also be present in carbon, nitrogen or mineral sources in the effective medium or can be added specifically to the medium.

The fermentation medium can also contain a suitable phosphate source. Such phosphate sources include both inorganic and organic phosphate sources. Preferred phosphate sources include, but are not limited to, phosphate salts such as mono or dibasic sodium and potassium phosphates, ammonium phosphate and mixtures thereof. Typically, the concentration of phosphate in the fermentation medium is greater than about 1.0 g/L, preferably greater than about 2.0 g/L and more preferably greater than about 5.0 g/L. Beyond certain concentrations, however, the addition of phosphate to the fermentation medium is not advantageous for the growth of the microorganisms. Accordingly, the concentration of phosphate in the fermentation medium is typically less than about 20 g/L, preferably less than about 15 g/L and more preferably less than about 10 g/L.

A suitable fermentation medium can also include a source of magnesium, preferably in the form of a physiologically acceptable salt, such as magnesium sulfate heptahydrate, although other magnesium sources in concentrations which contribute similar amounts of magnesium can be used. Typically, the concentration of magnesium in the fermentation medium is greater than about 0.5 g/L, preferably greater than about 1.0 g/L, and more preferably greater than about 2.0 g/L. Beyond certain concentrations, however, the addition of magnesium to the fermentation medium is not advantageous for the growth of the microorganisms. Accordingly, the concentration of magnesium in the fermentation medium is typically less than about 10 g/L, preferably less than about 5 g/L, and more preferably less than about 3 g/L. Further, in some instances it may be desirable to allow the fermentation medium to become depleted of a magnesium source during fermentation.

The fermentation medium can also include a biologically acceptable chelating agent, such as the dihydrate of trisodium citrate. In such instance, the concentration of a chelating agent in the fermentation medium is greater than about 0.2 g/L, preferably greater than about 0.5 g/L, and more preferably greater than about 1 g/L. Beyond certain concentrations, however, the addition of a chelating agent to the fermentation medium is not advantageous for the growth of the microorganisms. Accordingly, the concentration of a chelating agent in the fermentation medium is typically less than about 10 g/L, preferably less than about 5 g/L, and more preferably less than about 2 g/L.

The fermentation medium can also initially include a biologically acceptable acid or base to maintain the desired pH of the fermentation medium. Biologically acceptable acids include, but are not limited to, hydrochloric acid, sulfuric acid, nitric acid, phosphoric acid and mixtures thereof. Biologically acceptable bases include, but are not limited to, ammonium hydroxide, sodium hydroxide, potassium hydroxide and mixtures thereof. In a preferred embodiment of the present invention, the base used is ammonium hydroxide.

The fermentation medium can also include a biologically acceptable calcium source, including, but not limited to, calcium chloride. Typically, the concentration of the calcium source, such as calcium chloride, dihydrate, in the fermentation medium is within the range of from about 5 mg/L to about 2000 mg/L, preferably within the range of from about 20 mg/L to about 1000 mg/L, and more preferably in the range of from about 50 mg/L to about 500 mg/L.

The fermentation medium can also include sodium chloride. Typically, the concentration of sodium chloride in the fermentation medium is within the range of from about 0.1 g/L to about 5 g/L, preferably within the range of from about 1 g/L to about 4 g/L, and more preferably in the range of from about 2 g/L to about 4 g/L.

As previously discussed, the fermentation medium can also include trace metals. Such trace metals can be added to the fermentation medium as a stock solution that, for convenience, can be prepared separately from the rest of the fermentation medium. A suitable trace metals stock solution for use in the fermentation medium is shown below in Table 1. Typically, the amount of such a trace metals solution added to the fermentation medium is greater than about 1 ml/L, preferably greater than about 5 mL/L, and more preferably greater than about 10 mL/L. Beyond certain concentrations, however, the addition of a trace metals to the fermentation medium is not advantageous for the growth of the microorganisms. Accordingly, the amount of such a trace metals solution added to the fermentation medium is typically less than about 100 mL/L, preferably less than about 50 mL/L, and more preferably less than about 30 mL/L. It should be noted that, in addition to adding trace metals in a stock solution, the individual components can be added separately, each within ranges corresponding independently to the amounts of the components dictated by the above ranges of the trace metals solution.

As shown below in Table 1, a suitable trace metals solution for use in the present invention can include, but is not limited to ferrous sulfate, heptahydrate; cupric sulfate, pentahydrate; zinc sulfate, heptahydrate; sodium molybdate, dihydrate; cobaltous chloride, hexahydrate; and manganous sulfate, monohydrate. Hydrochloric acid is added to the stock solution to keep the trace metal salts in solution.

TABLE 1
Trace Metals Stock Solution

| | CONCENTRATION |
|---|---|
| COMPOUND | (mg/L) |
| Ferrous sulfate heptahydrate | 280 |
| Cupric sulfate, pentahydrate | 80 |
| Zinc (II) sulfate, heptahydrate | 290 |
| Sodium molybdate, dihydrate | 240 |
| Cobaltous chloride, hexahydrate | 240 |
| Manganous sulfate, monohydrate | 170 |
| Hydrochloric acid | 0.1 ml |

The fermentation medium can also include vitamins. Such vitamins can be added to the fermentation medium as a stock solution that, for convenience, can be prepared separately from the rest of the fermentation medium. A suitable vitamin stock solution for use in the fermentation medium is shown below in Table 2. Typically, the amount of such vitamin solution added to the fermentation medium is greater than 1 ml/L, preferably greater than 5 ml/L and more preferably greater than 10 ml/L. Beyond certain concentrations, however, the addition of vitamins to the fermentation medium is not advantageous for the growth of the microorganisms. Accordingly, the amount of such a vitamin solution added to the fermentation medium is typically less than about 50 ml/L, preferably less than 30 ml/L and more preferably less than 20 ml/L. It should be noted that, in addition to adding vitamins in a stock solution, the individual components can be added separately each within the ranges corresponding independently to the amounts of the components dictated by the above ranges of the vitamin stock solution.

As shown in Table 2, a suitable vitamin solution for use in the present invention can include, but is not limited to, biotin, calcium pantothenate, inositol, pyridoxine-HCl and thiamine-HCl.

TABLE 2
Vitamin Stock Solution

| COMPOUND | CONCENTRATION (mg/L) |
|---|---|
| Biotin | 10 |
| Calcium pantothenate | 120 |
| Inositol | 600 |
| Pyridoxine-HCl | 120 |
| Thiamine-HCl | 120 |

As stated above, when an organism is blocked in the sterol pathway, an exogenous sterol must be added to the fermentation medium. Such sterols include, but are not limited to, ergosterol and cholesterol. Such sterols can be added to the fermentation medium as a stock solution that is prepared separately from the rest of the fermentation medium. Sterol stock solutions can be prepared using a detergent to aid in solubilization of the sterol. Typically, an amount of sterol stock solution is added to the fermentation medium such that the final concentration of the sterol in the fermentation medium is within the range of from about 1 mg/L to about 3000 mg/L, preferably within the range from about 2 mg/L to about 2000 mg/L, and more preferably within the range from about 5 mg/L to about 2000 mg/L.

Microorganisms of the present invention can be cultured in conventional fermentation modes, which include, but are not limited to, batch, fed-batch, cell recycle, and continuous. It is preferred, however, that the fermentation be carried out in fed-batch mode. In such a case, during fermentation some of the components of the medium are depleted. It is possible to initiate the fermentation with relatively high concentrations of such components so that growth is supported for a period of time before additions are required. The preferred ranges of these components are maintained throughout the fermentation by making additions as levels are depleted by fermentation. Levels of components in the fermentation medium can be monitored by, for example, sampling the fermentation medium periodically and assaying for concentrations. Alternatively, once a standard fermentation procedure is developed, additions can be made at timed intervals corresponding to known levels at particular times throughout the fermentation. As will be recognized by those in the art, the rate of consumption of nutrient increases during fermentation as the cell density of the medium increases. Moreover, to avoid introduction of foreign microorganisms into the fermentation medium, addition is performed using aseptic addition methods, as are known in the art. In addition, a small amount of anti-foaming agent may be added during the fermentation.

The temperature of the fermentation medium can be any temperature suitable for growth and production of farnesol or GG. For example, prior to inoculation of the fermentation medium with an inoculum, the fermentation medium can be brought to and maintained at a temperature in the range of from about 20° C. to about 45° C., preferably to a temperature in the range of from about 25° C. to about 40° C., and more preferably in the range of from about 28° C. to about 32° C.

The pH of the fermentation medium can be controlled by the addition of acid or base to the fermentation medium. In such cases when ammonia is used to control pH, it also conveniently serves as a nitrogen source in the fermentation medium. Preferably, the pH is maintained from about 3.0 to about 8.0, more preferably from about 3.5 to about 7.0, and most preferably from about 4.0 to about 6.5.

The fermentation medium can also be maintained to have a dissolved oxygen content during the course of fermentation to maintain cell growth and to maintain cell metabolism for production of farnesol or GG. The oxygen concentration of the fermentation medium can be monitored using known methods, such as through the use of an oxygen electrode. Oxygen can be added to the fermentation medium using methods known in the art, for example, through agitation and aeration of the medium by stirring, shaking or sparging. Preferably, the oxygen concentration in the fermentation medium is in the range of from about 20% to about 100% of the saturation value of oxygen in the medium based upon the solubility of oxygen in the fermentation medium at atmospheric pressure and at a temperature in the range of from about 20° C. to about 40° C. Periodic drops in the oxygen concentration below this range may occur during fermentation, however, without adversely affecting the fermentation.

Although aeration of the medium has been described herein in relation to the use of air, other sources of oxygen can be used. Particularly useful is the use of an aerating gas which contains a volume fraction of oxygen greater than the volume fraction of oxygen in ambient air. In addition, such aerating gases can include other gases which do not negatively affect the fermentation.

In an embodiment of the fermentation process of the present invention, a fermentation medium is prepared as described above. This fermentation medium is inoculated with an actively growing culture of microorganisms of the present invention in an amount sufficient to produce, after a reasonable growth period, a high cell density. Typical inoculation cell densities are within the range of from about 0.01 g/L to about 10 g/L, preferably from about 0.2 g/L to about 5 g/L and more preferably from about 0.05 g/L to about 1.0 g/L, based on the dry weight of the cells. In production scale fermentors, however, greater inoculum cell densities are preferred. The cells are then grown to a cell density in the range of from about 10 g/L to about 100 g/L preferably from about 20 g/L to about 80 g/L, and more preferably from about 50 g/L to about 70 g/L. The residence times for the microorganisms to reach the desired cell densities during fermentation are typically less than about 200 hours, preferably less than about 120 hours, and more preferably less than about 96 hours.

In one mode of operation of the present invention, the carbon source concentration, such as the glucose concentration, of the fermentation medium is monitored during fermentation. Glucose concentration of the fermentation medium can be monitored using known techniques, such as, for example, use of the glucose oxidase enzyme test or high pressure liquid chromatography, which can be used to monitor glucose concentration in the supernatant, e.g., a cell-free component of the fermentation medium. As stated previously, the carbon source concentration should be kept below the level at which cell growth inhibition occurs. Although such concentration may vary from organism to organism, for glucose as a carbon source, cell growth inhibition occurs at glucose concentrations greater than at about 60 g/L, and can be determined readily by trial. Accordingly, when glucose is used as a carbon source the glucose is preferably fed to the fermentor and maintained below detection limits. Alternatively, the glucose concentration in the fermentation medium is maintained in the range of from about 1 g/L to about 100 g/L, more preferably in the range of from about 2 g/L to about 50 g/L, and yet more preferably in the range of from about 5 g/L to about 20 g/L. Although the carbon source concentration can be maintained within desired levels by addition of, for example, a substantially pure glucose solution, it is acceptable, and may be preferred, to maintain the carbon source concentration of the fermentation medium by addition of aliquots of the original fermentation medium. The use of aliquots of the original fermentation medium may be desirable because the concentrations of other nutrients in the medium (e.g. the nitrogen and phosphate sources) can be maintained simultaneously. Likewise, the trace metals concentrations can be maintained in the fermentation medium by addition of aliquots of the trace metals solution.

3.3 Farnesol and GG Recovery

Once farnesol or GG are produced by a biological system, they are recovered or isolated for subsequent use. The present inventors have shown that for both farnesol and GG, the product may be present in culture supernatants and/or associated with the yeast cells. With respect to the cells, the recovery of farnesol or GG includes some method of permeabilizing or lysing the cells. The farnesol or GG in the culture can be recovered using a recovery process including, but not limited to, chromatography, extraction, solvent extraction, membrane separation, electrodialysis, reverse osmosis, distillation, chemical derivatization and crystallization. When the product is in the phosphate form, i.e., farnesyl phosphate or geranylgeranyl phosphate, it only occurs inside of cells and therefore, requires some method of permeabilizing or lysing the cells.

4. Enzymatic Production of Geranylgeraniol

Another method of biological production of geranylgeraniol is by an enzymatic production process using isopentyl diphosphate as an initial substrate.

4.1 Cell-Free Production of Geranylgeraniol from IPP, IDI and GGPP Synthase

GGOH can be produced enzymatically by incubating isopentyl diphosphate (IPP) in the presence of dimethylallyl diphosphate (DMAPP) isomerase (IDI) and geranylgeraniol diphosphate (GGPP) synthase. This reaction is described, for example, in Huang et al., Tetrahedron Letters, 39, 2033-2036 (1998), which is incorporated herein by reference in its entirety. IDI can be prepared by expression of the Schizosaccharomyces pombe IPP isomerase cDNA clone in Escherichia coli by known methods and GGPP synthase is obtained by expression of the bacterial crtE gene cloned into Escherichia coli. See Hahn and Poulter, J. Biol. Chem. 270 (19), 11298 (1995); Math et al., Proc. Natl. Acad. Sci. USA, 89, 6761(1992); U.S. Pat. No. 5,912,154; and U.S. Pat. No. 5,766,911. In this reaction, IDI catalyzes the 1,3-allylic rearrangement reaction converting IPP to its electrophilic isomer DMAPP. These two isomers are initial substrates for prenyltransferases that synthesize polyisoprenoid chains. GGPP synthase combines IPP with DMAPP to form GGPP. The concentrations of IPP and DMAPP are regulated by IDI such that substantially all of the IPP starting material is consumed. The inclusion of an inorganic pyrophosphatase improves the efficiency of GGPP synthase by removing product inhibition. If a phosphatase is not included in the reaction, one must be used later to dephosphorylate the GGPP produced to geranylgeraniol. The reaction is typically incubated overnight at physiological pH and temperature and alternative reaction conditions are within the skill of those in the art.

4.2 Cell-Free Production of Geranylgeraniol without IDI

GGOH can also be produced in the absence of IDI by combining IPP with a compound selected from the group consisting of DMAPP, geranyl diphosphate (GPP), farnesyl diphosphate (FPP) and mixtures thereof and incubating in the presence of GGPP synthase. This reaction is described in Huang et al. In the presence of excess DMAPP, GPP and/or FPP, residual unreacted IPP remains after lengthy incubation such that careful selection of the molar ratios of the starting materials is useful to ensure the most efficient GGPP production. This reaction is also incubated overnight at physiological pH and temperature, and an inorganic pyrophosphatase can be added to increase the efficiency of the GGPP synthase or a phosphatase is subsequently used to dephosphorylate the GGPP produced to geranylgeraniol.

4.3 Production of IPP Starting Material

IPP is a starting material in the two processes described above in Sections 4.1 and 4.2 and can be produced by known methods. For example, as described in Huang et al., 1-hydroxy-3-butanone is produced by an aldol condensation between acetone and formaldehyde at about pH 10. See Hays et al., J Am. Soc. Chem. 73, 5369 (1951). The butanone is then activated and bisphosphorylated to produce IPP. See Davidson et al., J. Org. Chem. 51, 4768 (1986).

5.0 Chemical Synthesis of Teprenone from Geranylgeraniol

In a further embodiment of the present invention, a method for producing teprenone from geranylgeraniol is provided. Geranylgeraniol contains four olefin moieties. As used in this invention, the olefin moieties of geranylgeraniol are consecutively numbered starting from the hydroxy terminus, i.e., the first olefin moiety refers to C₂-C₃ double bond, the second olefin moiety refers to C₆-C₇ double bond, the third olefin moiety refers to C₁₀-C₁₁ double bond and the fourth olefin moiety refers to C₁₄-C₁₅ double bond.

In the first step of the synthesis of teprenone by the method of the present invention, geranylgeraniol (GGOH) is converted to an alkyl halide. This is done by reacting GGOH with a halogenating reagent to convert the allylic alcohol of GGOH to an allylic halide. As used in this invention, a halogenating reagent is any chemical used to convert alcohols into alkyl halides without rearrangement. These reagents typically undergo reaction with alcohols to form inorganic esters which are good leaving groups. Phosphorous trihalides such as PF₃, PCl₃, PBr₃, or PI₃ and thionyl halides such as SOF₂, SOCL₂, SOBr₂ or SOI₂ can be used to perform this conversion. A phosphorus trichloride undergoes reaction with an alcohol to yield a phosphite ester and HCl. This initial reaction step does not involve cleavage of the carbon-oxygen bond. No racemization of a pure enantiomeric alcohol is observed, as would happen if the reaction went through a carbocation. The second step in the reaction is the S_{N}2 attack by Cl⁻. Each of the three halide-phosphorus bonds can undergo reaction and the end product is phosphorous acid (H₃PO₃) and halogenated geranylgeranyl. Thionyl chloride can also be used as a halogenating reagent. In this case, if an amine solvent is used for the reaction, a chiral, resolved alcohol yields the alkyl chloride with the inverted configuration. Conversely, if an ether solvent is used, the alkyl chloride that is formed has the same configuration as that of the starting alcohol. In either solvent, the first step of the reaction sequence is analogous to that of the reaction with phosphorus trihalide; the formation of an inorganic ester. Again the carbon-oxygen bond is not broken in this first step. If the starting alcohol is a pure enantiomer, the chlorosulfite ester has the same configuation as the alcohol. An amine solvent reacts with the HCl formed in this reaction to yield an amine salt. The chloride ion from this acid-base reaction attacks the chlorosulfite ester in a typical S_{N}2 reaction, which results in a alkyl chloride with an inverted configuration. Preferably, the halogenating reagent is PBr₃ resulting in the production of geranylgeranyl bromide (3,7,11,15-tetramethyl-2,6,10,14-hexadecatetraene 1-bromide).

The step of forming an alkyl halide can be conducted under standard reaction conditions that would be known to those skilled in the art. For example, PBr₃ is added to the geranylgeraniol in a solvent at −20° C. under inert gas. The reaction is stirred and warmed to room temperature before being neutralized with a base such as cold bicarbonate and the solvent is allowed to evaporate.

The next step in this embodiment converts the geranylgeranyl halide to a keto ester intermediate. An alkyl acetoacetate is added to the halide in the presence of a base to form the intermediate. Suitable bases include amines in an aqueous solvent, wherein the base is present in an amount of about 1 to about 20 mole percent of the geranylgeranyl halide. Specific examples of suitable bases include, but are not limited to, primary amines such as butylamine, hexylamine, tetradecylamine, stearylamine, and ethylenediamine, secondary amines such as diethylamine, dipropylamine, ethylpropylamine, and diethanolamine, tertiary amines such as triethylamine, dimethylpropylamine, and triethanolamine, and quaternary ammonium salts such as tetrapentyl ammonium salt, lauryldimethylethylammonium salt and benzyltrimethylammonium salt. The halide readily attacks the carbon alpha to the ketone and the ester to release a halide ion and water. An enolate ion is formed when hydroxide ions combine with a hydrogen of the alpha carbon to form water. The enolate ion then quickly undergoes reaction with the halide to yield the keto ester intermediate and a halide ion. The mole ratio of the alkyl acetoacetate to geranylgeranyl halide can be from 1:2 to 30:1, preferably about 3:1 to 10:1. The halide is added to a cooled solution of alkyl acetoacetate at a temperature of less than 20° C., preferably less than 5° C., and incubated for greater than 6 hours to complete the reaction.

Preferred alkyl acetoacetates include methyl acetoacetate, ethyl acetoacetate, propyl acetoacetate and butyl acetoacetate, with ethyl acetoacetate being particularly preferred.

The final step in this embodiment is to remove the ester group from the keto ester intermediate to produce teprenone. This step is conducted in the presence of an alkali metal hydroxide such as NaOH or KOH. The ester group is removed and the keto ester intermediate undergoes hydrolysis and decarboxylation to form teprenone and carbon dioxide. The alkali metal hydroxide is typically present at about 0.3 mole per mole of water in the reaction or greater. The preferred amount is about 2.5 to about 4 moles per mole of geranylgeranyl halide. This reaction is carried out in the temperature range of about 0° C. to about 150° C., preferably in the range of about 40° C. to about 80° C., under refluxing at atmospheric conditions. The reaction is continued until nearly all of the geranylgeranyl halide is consumed which occurs between about 10 minutes and about 40 hours.

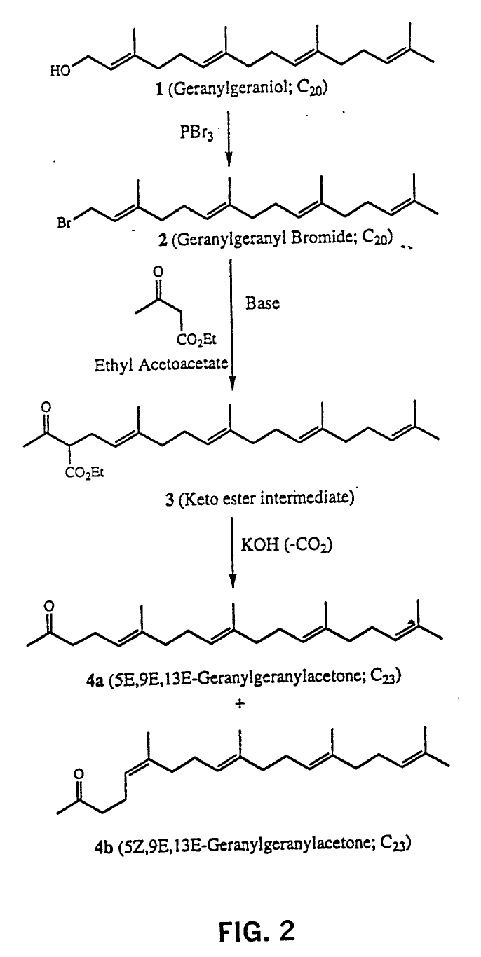

With reference to FIG. 2, a preferred process for chemical synthesis of teprenone from geranylgeraniol is shown. More particularly, geranylgeraniol is reacted with PBr3 to produce the alkyl halide geranylgeranyl bromide. Geranylgeranyl bromide is then reacted with ethyl acetoacetate under basic conditions to form a keto ester intermediate, which is reacted with KOH to form teprenone. This process substantially retains the isomeric form of the geranylgeraniol starting material. Thus, to the extent that the geranylgeraniol starting material is predominantly the 2E isomer, the resulting teprenone will be predominantly 5E,9E,13E,17-geranylgeranylacetone.

The method of the present invention is particularly advantageous over other known methods of producing teprenone because the present method can result in the production of teprenone which approaches 100% of the 5E-isomer (6,10,14,18-tetramethyl-5E,9E,13E,17-nonadecatetraen-2-one). This ability to produce predominately the 5E-isomer of teprenone is achieved by obtaining geranylgeraniol from an isomerically pure source, such as a biological service, and then chemically converting that material to teprenone via mechanisms that retain the isomeric configuration of the starting material. Preferably, teprenone produced by the method of the present invention contains at least about 75% of the 5E isomer, and more preferably at least about 80%, and more preferably at least about 85%, and more preferably at least about 90%, and more preferably at least about 95%, and more preferably at least about 96%, and more preferably at least about 97%, and more preferably at least about 98%, and more preferably at least about 99%, and even more preferably at least about 99.9%, and most preferably 100% of the isomer.

Various methods known to those of skill in the art can be used to carry out the chemical steps claimed. It is to be understood that the present invention contemplates and encompasses any and all conservative substitutions in the chemical formulas and reactions recited which result in the production of the 5E isomer of teprenone from geranylgeraniol obtained from a biological source, and preferably, teprenone which approaches 100% of the 6,10,14,18-tetramethyl-5E,9E,13E,17-nonadecatetraen-2-one isomer.

The following Examples are provided to illustrate embodiments of the present invention and are not intended to limit the scope of the invention as set forth in the claims.

### EXAMPLES

### Example 1

The following example shows one means of increasing the activity of FPP phosphatase in microorganisms capable of producing GGPP and the effect on the phosphatase activity in these cells.

The activity of the Dpp1 phosphatase was increased in strains that carry the erg9 mutation by elevating the DPP1 gene copy number. The DPP1 gene, including its native promoter, was amplified by PCR using genomic DNA from strain S288C (Yeast Genetic Stock Center, Berkeley, Calif.) and the following oligonucleotides.

| | |
|---|---|
| Oligo Name | Oligo Sequence |

| | | |
|---|---|---|
| VE149-5 | ctgtgaagctcgcatactctgcagataatcag | (SEQ ID NO:1) |
| VE150-3 | gtcagtaaagtcgaccatataaatggaacgtatcgc | (SEQ ID NO:2) |

The amplified DPP1 gene was then cloned into the high copy-number yeast plasmid YEp352 (Hill et al., 1986. YEAST 2:163-167) to generate plasmid pTWM144. This plasmid was used to transform the erg9 mutant strain SW23B#74 (U.S. Pat. No. 6,242,227), resulting in the strain referred to as CALI3. Elevation of farnesyl pyrophosphate (FPP) phosphatase activity was demonstrated using cell extracts prepared from CALI3, and compared to cell extracts prepared from SW23B#74. FPP phosphatase activity was measured as follows. Cells were grown to OD₆₀₀=2.0, then harvested by centrifugation at 2600 rpm for 10 min. The cell pellets were resuspended in breaking buffer (10 mM TRIS, 1 mM EDTA, 1 mM dithiothreitol, pH 7.5), and 0.5 mm zirconium silica beads were added. The cell suspensions were agitated in a bead beater for six 1-minute cycles, cooling on ice between each cycle. The broken cell suspensions were transferred to centrifuge tubes and spun at 1000×g for 5 minutes. The supernatants were recovered and used as the cell extracts for enzyme assays.

FPP phosphatase assay was carried out in 25 mM BIS-TRIS-PROPANE buffer, pH 7.0, 0.1% triton X-100, 57.5 uM farnesyl pyrophosphate, and varying amounts of cell extract. The reaction was incubated at 35° C. for 30 minutes, and then terminated by the addition of an equal volume of methanol and an equal volume of hexane. The mixture was vortexed vigorously for 3 minutes, centrifuged at 2600 rpm for 10 min, and the hexane layer was assayed by GC to determine the amount of farnesol formed from FPP.

| | FPP Phosphatase Activity |
|---|---|
| | nmol FOH formed/ |
| Strain | (mg protein × min) |
| SW23B#74 (control) | 1.36 × 10⁻⁴ |
| CALI3-1 (elevated DPP1) | 7.47 × 10⁻⁴ |

This example demonstrates that isoprenoid phosphatase activity can be increased by overexpression of the DPP1 gene.

### Example 2

The following example describes one means of decreasing the activity of FPP phosphatase in a microorganism capable of producing GGPP and the effect on the measured phosphatase activity.

The activity of the Dpp1 phosphatase was decreased in strains that carry the erg9 mutation by introducing a dpp1 deletion mutation. This was accomplished by constructing a deletion allele of the DPP1 gene in the yeast integrating vector pRS306 (Sikorski and Hieter, 1989, Genetics 122:19-27), and then using that plasmid to disrupt the chromosomal DPP1 gene using a pop-in/pop-out gene replacement method (Rothstein, 1991, Meth. Enzymology, 194:293-298). To construct the deletion allele, the DPP1 gene (see Accession No. NC001136) was ligated into the SacI and SalI sites in pRS306 to create plasmid pCALI 1. This plasmid was then digested with BsrGI and BamHI to remove a 1.17 kb fragment from within the DPP1 gene. The remainder of the plasmid was re-ligated to create plasmid pCALI2, which was then used to transform the erg9 mutant strain SW23B#74. The pop-in/pop-out gene replacement method was then used to isolate strains that carried only the dpp1 deletion allele. One of the resulting strains that carried the erg9 and dpp1 mutations was referred to as CALI5- 1.

Reduction of FPP phosphatase activity was demonstrated using cell extracts prepared from CALI5, and compared to cell extracts prepared from SW23B#74. FPP phosphatase activity was measured as described above.

| | FPP Phosphatase Activity |
|---|---|
| | nmol FOH formed/ |
| Strain | (mg protein × min) |
| SW23B#74 (control) | 1.36 × 10⁻⁴ |
| CALI5-1 (dpp1 mutant) | 0.14 × 10⁻⁴ |

The foregoing description of the invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiments described herein above are further intended to explain best modes known of practicing the invention and to enable others skilled in the art to utilize the invention in such, or other, embodiments and with the various modifications required by the particular applications or uses of the invention. It is intended that the appended claims be construed to include alternative embodiments to the extent permitted by the prior art.

What is claimed is:

1. A method of making teprenone comprising:
(a) reacting geranylgeraniol with an alkyl acetoacetate to form a keto ester intermediate; and,
(b) decarboxylating said intermediate to form teprenone.

2. The method of claim 1, wherein said reacting step comprises:
(a) reacting geranylgeraniol with a halogenating reagent to form an alkyl halide; and,
(b) reacting said alkyl halide with said alkyl acetoacetate in the presence of a base to form said keto ester intermediate.

3. The method of claim 2, wherein said halogenating reagent is selected from a member of the group consisting of PF₃, PCl₃, PBr₃, PI₃, SOF₂, SOCl₂, SOBr₂, and SOI₂.

4. The method of claim 2, wherein said halogenating reagent is PBr₃ and the alkyl halide formed is geranylgeranyl bromide.

5. The method of claim 2, wherein said alkyl acetoacetate is selected from a member of the group consisting of methylacetoacetate, ethylacetoacetate, propylacetoacetate, and butylacetoacetate.

6. The method of claim 2, wherein said alkyl acetoacetate is ethylacetoacetate.

7. The method of claim 2, wherein said base is selected from the group consisting of primary amines, secondary amines, tertiary amines, quaternary ammonium salts.

8. The method of claim 1, wherein said decarboxylating step comprises treating said keto ester intermediate with an alkaline reagent.

9. The method of claim 2, wherein said alkaline reagent is selected from the group consisting of an aqueous sodium hydroxide solution and an aqueous potassium hydroxide solution.

10. The method of claim 1, wherein said compound formed contains at least 75% of the 6,10,14,18-tetramethyl-5E,9E,13E,17E-nonadecatetraen-2-one isomer.

11. The method of claim 1, wherein said compound formed contains at least 90% of the 6,10,14,18-tetramethyl-5E,9E,13E,17E-nonadecatetraen-2-one isomer.

12. The method of claim 1, wherein said compound formed contains at least 95% of the 6,10,14,18-tetramethyl-5E,9E,13E,17E-nonadecatetraen-2-one isomer.

13. The method of claim 1, wherein geranylgeraniol is produced biologically.

14. A method of making teprenone comprising:
(a) biologically producing geranylgeraniol;
(b) reacting said geranylgeraniol with a halogenating reagent to form an alkyl halide;
(c) reacting said alkyl halide with said alkyl acetoacetate in the presence of a base to form said keto ester intermediate; and,
(d) decarboxylating said intermediate to form teprenone.

15. The method of claim 14, wherein geranylgeraniol is produced by a process comprising:
(a) reacting isopentyl diphosphate with isopentenyl diphosphate:dimethylallyl diphosphate isomerase, in the presence of geranylgeranyl diphosphate synthase to form geranylgeranyl diphosphate; and,
(b) dephosphorylating said geranylgeranyl diphosphate to obtain geranylgeraniol.

16. The method of claim 14, wherein geranylgeraniol is produced by a process comprising:
(a) reacting isopentyl diphosphate with a compound selected from the group consisting of dimethylallyl diphosphate, geranyl diphosphate, and farnesyl diphsophate, in the presence of geranylgeranyl diphosphate synthase to form geranylgeranyl diphosphate; and,
(b) dephosphorylating said geranylgeranyl diphosphate to obtain geranylgeraniol.

17. The method of claim 14, wherein geranylgeraniol is produced by a process comprising:
(a) culturing a microorganism in a fermentation medium to produce geranylgeraniol; and,
(b) recovering said geranylgeraniol.

18. The method of claim 17, wherein said microorganism is genetically modified to decrease the activity of squalene synthase.

19. The method of claim 17, wherein said microorganism is further genetically modified to increase the activity of HMG-CoA reductase.

20. The method of claim 19, wherein the activity of HMG-CoA reductase is increased by overexpression of HMG-CoA reductase or the catalytic domain thereof in the microorganism.

21. The method of claim 20, wherein said microorganism is further genetically modified to increase the activity of a protein selected from the group consisting of acetoacetyl Co-A thiolose, HMG-CoA synthase, mevalonate kinase, phosphomevalonate kinase, phosphomevalonate decarboxylase, isopentenyl pyrophosphate isomerase, farnesyl pyrophosphate synthase, D-1-deoxyxylulose 5-phosphate synthase, and 1-deoxy-D-xylulose 5-phosphate reductoisomerase.

22. The method of claim 17, wherein the microorganism has been genetically modified to increase the activity of farnesyl pyrophosphate synthase.

23. The method of claim 17, wherein the microorganism has been genetically modified to increase the activity of an isoprenoid phosphatase.

24. The method of claim 17, wherein the microorganism has been genetically modified to increase the activity of geranylgeraniol diphosphate synthase.

25. The method of claim 17, wherein said microorganism is an erg9 mutant.

26. The method of claim 17, wherein said geranylgeraniol is secreted into said fermentation medium by said microorganism and wherein said step of recovering comprises purification of said geranylgeraniol from said fermentation medium.

27. The method of claim 17, wherein said step of recovering comprises isolating said geranylgeraniol from said microorganism.

28. The method of claim 17, wherein said step of culturing produces geranylgeranyl pyrophosphate and said step of recovering further comprises dephosphorylating said geranylgeranyl pyrophosphate to produce geranylgeraniol.

29. A method of making teprenone comprising:
(a) culturing a microorganism in a fermentation medium to produce geranylgeraniol;
(b) reacting said geranylgeraniol with a halogenating reagent to form an alkyl halide;
(c) reacting said alkyl halide with said alkyl acetoacetate in the presence of a base to form a keto ester intermediate,
(d) decarboxylating said keto ester intermediate to form teprenone, wherein said teprenone comprises greater than 90% 5E,9E,13E-Geranylgeranylacetone.
